(19) United States
(12) Patent Application Publication
(10) Pub. No.: US 2026/0355735 A1
(43) Pub. Date: Nov. 12, 2026

(54) FLUID TRANSFER SYSTEM, METHOD FOR FLUID TRANSFER, AND BIOCHEMICAL SUBSTANCE ANALYSIS SYSTEM

(71) Applicant: MGI Tech Co., Ltd., Shenzhen (CN)

(72) Inventors: ZI-HUA NIU, Shenzhen (CN); HAO LU, Shenzhen (CN); XIAO-LI ZHOU, Shenzhen (CN); CHUTIAN XING, Shenzhen (CN)

(21) Appl. No.: 19/514,988

(22) PCT Filed: Sep. 14, 2023
(86) PCT No.: PCT/CN2023/118746

§ 371 Date: Feb. 27, 2026

(51) Int. Cl. B01L 3/00

(52) U.S. Cl. CPC B01L 3/502; B01L 2200/026; B01L 2300/14; B01L 2400/0633

(57) ABSTRACT

A fluid transfer system, a method for fluid transfer, and a biochemical substance analysis system are provided. The fluid transfer system includes a fluid storage module used to store fluid, a fluid utilization module with a flow channel, a quantitative fluid driving module, and a pressure control module. The pressure control module is connected to the fluid storage module. The pressure control module applies a pressure to the fluid storage module and maintains a positive pressure state at an inlet of the flow channel. The quantitative fluid driving module generates a driving force to quantitatively transfer fluid from the storage module to the flow channel. This configuration reduces gas bubble formation, improves reaction quality, enhances transfer accuracy, simplifies structure, lowers cost, allows flexible flow rate control, minimizes cross-contamination, and ensures consistent fluid volumes.

### FIELD

The subject matter relates to the technical field of biochemical substance analysis, and more particularly, to a fluid transfer system, a method for fluid transfer, and a biochemical substance analysis system.

### BACKGROUND

In fields of biology, chemistry, and medicine, instruments, such as gene sequencers, are often designed based on core principles of biological or chemical reactions. Substances participating in these reactions (such as reagents or biological samples) are typically in a liquid or gaseous state, which may be referred to as fluids. To perform required biological or chemical reactions within the instrument, a container (such as sequencing chip) is often used as reaction chamber for these biochemical substances. Furthermore, the instrument requires a fluid transfer system capable of quantitatively transporting different fluids into the reaction chamber according to predetermined requirements.

When designing the fluid transfer system, it is necessary to develop fluidic components with suitable performance and design a rational flow layout and operational logic to meet various requirements of the biological or chemical reactions within the instrument. And the requirements relate to various aspects including fluid transport speed, pressure, precision, accuracy, and flow pulsation. However, the existing fluid transfer systems may have complex structures, high equipment costs, limited fluid flow rates, and low fluid transfer efficiency. Moreover, gas bubbles can generate during liquid transfer of the fluid transfer system, which adversely affect the quality of biochemical reactions.

### SUMMARY

In order to solve at least one problem above and/or other potential problems in the related art, a fluid transfer system and a method for fluid transfer are necessarily proposed.

Additionally, a biochemical substance analysis system with the fluid transfer system is also provided in an embodiment of the present disclosure.

In a first aspect, a fluid transfer system is provided. The fluid transfer system includes a fluid storage module, a fluid utilization module, a quantitative fluid driving module, and a pressure control module. The fluid storage module is used to store fluid. The fluid utilization module defines a flow channel, wherein the flow channel comprises an inlet and an outlet. The inlet of the flow channel is connected to the fluid storage module, the outlet of the flow channel is in connection with the quantitative fluid driving module. The fluid storage module is also connected to the pressure control module. Wherein the pressure control module is configured to apply pressure to the fluid storage module and to maintain a positive pressure state at the inlet of the flow channel, and the quantitative fluid driving module is configured to generate a driving force to quantitatively transfer the fluid from the fluid storage module to the flow channel.

In some possible embodiments, the fluid storage module comprises a fluid storage device and a fluid temporary storage device connected to the fluid storage device, and each of the pressure control module and the flow channel is connected to the fluid temporary storage device; wherein the pressure control module is configured to apply the pressure to the fluid temporary storage device, the pressure comprises a negative pressure and a positive pressure, the negative pressure is configured to transfer the fluid from the fluid storage device to the fluid temporary storage device, and the positive pressure is applied to the fluid temporary storage device and configured to maintain the positive pressure state at the inlet of the flow channel.

In some possible embodiments, the fluid storage module comprises a fluid storage device, the fluid storage device is connected to each of the pressure control module and the flow channel; wherein the pressure control module is configured to apply the pressure to the fluid storage device, the pressure a positive pressure, the positive pressure is configured to apply to the fluid storage device and maintain the positive pressure state at the inlet of the flow channel.

In some possible embodiments, the pressure control module comprises at least one pressure source, at least one pressure monitoring device, and at least one switching valve. The pressure source being connected to the fluid storage device or to the fluid temporary storage device. The pressure monitoring device configured to monitor a pressure applied to the at least one pressure source. The at least one switching valve disposed between the at least one pressure source and the fluid storage device or between the at least one pressure source and the fluid temporary storage device.

In some possible embodiments, wherein the pressure control module further comprises at least one gas reservoir disposed between the at least one pressure source and the at least one switching valve, the at least one gas reservoir is connected to the at least one pressure source and the fluid storage device or connected to the at least one pressure source and the fluid temporary storage device.

In some possible embodiments, wherein the at least one pressure source comprises a positive pressure source, or the at least one pressure source comprises a negative pressure source and a positive pressure source; and the at least one gas reservoir comprises a positive pressure gas reservoir, or the at least one gas reservoir comprises a negative pressure gas reservoir and a positive pressure gas reservoir, the positive pressure gas reservoir is connected to the positive pressure source, and the negative pressure gas reservoir is connected to the negative pressure source.

In some possible embodiments, wherein the quantitative fluid driving module comprises a fluid driving device in connection with the flow channel, wherein the fluid driving device is configured to generate the driving force as a negative pressure driving force to quantitatively transfer the fluid from the fluid storage module to the flow channel.

In some possible embodiments, wherein the fluid driving device is further configured to generate the driving force as a positive pressure driving force to quantitatively transfer the fluid from the flow channel to the fluid storage module or to quantitatively transfer the fluid from the flow channel into a pipeline between the flow channel and the fluid storage module.

In some possible embodiments, the fluid driving device is a hydraulic unit or a pressure power unit.

In some possible embodiments, the hydraulic unit is a peristaltic pump, a plunger pump, a syringe pump, a gear pump, or a diaphragm pump; and the pressure power unit is a vacuum pump, a pressure pipettor, a diaphragm pump, or an air compressor.

In some possible embodiments, the fluid transfer system further includes a waste liquid storage module, wherein the quantitative fluid driving module is further configured to generate a driving force to transfer a reacted fluid from the flow channel to the waste liquid storage module.

In some possible embodiments, the waste liquid storage module is connected to the quantitative fluid driving module, the quantitative fluid driving module is configured to generate the driving force as a negative pressure driving force to transfer the reacted fluid from the flow channel to the quantitative fluid driving module, and the quantitative fluid driving module is further configured to generate the driving force as a positive pressure driving force to transfer the reacted fluid from the quantitative fluid driving module to the waste liquid storage module.

In some possible embodiments, the quantitative fluid driving module comprises a passage, and the fluid utilization module comprises at least one flow channel, the at least one flow channel is connected to the passage; or the quantitative fluid driving module comprises a plurality of passages, and the fluid utilization module comprises a plurality of flow channels, each of the plurality of flow channels is connected to a corresponding one of the plurality of passages, and each of the plurality of passages is configured to be controlled independently or controlled in a coordinated manner with other passages of the plurality of passages.

In some possible embodiments, a size of each of the plurality of flow channels is same, such that a time for the fluid to flow from the fluid storage module into each of the plurality of flow channels is same.

In some possible embodiments, the fluid temporary storage device comprises at least one temporary storage pool, and each of the pressure control module, the fluid storage device, and the flow channel is connected to the at least one temporary storage pool.

In some possible embodiments, the fluid storage device comprises at least one container for holding the fluid.

In some possible embodiments, the fluid utilization module is a biochemical reaction module configured to adjust a temperature of the flow channel to perform a biochemical reaction of the fluid transferred into the flow channel.

In a second aspect, a method for fluid transfer is provided. The method includes: apply a pressure to a fluid storage module through a pressure control module, and maintain a positive pressure state at an inlet of a flow channel of a fluid utilization module; and generate a driving force by a quantitative fluid driving module to quantitatively transfer fluid from the fluid storage module into the flow channel.

In some possible embodiments, the fluid storage module comprises a fluid storage device and a fluid temporary storage device connected to the fluid storage device, and each of the pressure control module and the flow channel is connected to the fluid temporary storage device; wherein applying the pressure to the fluid storage module comprises: applying a negative pressure to the fluid temporary storage device through the pressure control module to transfer fluid from the fluid storage device to the fluid temporary storage device; and applying a positive pressure to the fluid temporary storage device through the pressure control module and maintaining the positive pressure state at the inlet of the flow channel.

In some possible embodiments, the fluid storage module includes a fluid storage device connected to each of the pressure control module and the flow channel; wherein apply the pressure to the fluid storage module includes: apply a positive pressure to the fluid storage device by the pressure control module, and maintain a positive pressure state at the inlet of the flow channel.

In some possible embodiments, the quantitative fluid driving module comprises a fluid driving device connected to the flow channel; wherein generate the driving force includes: generate the driving force as a negative pressure driving force by the fluid driving device to quantitatively transfer the fluid from the fluid storage module to the flow channel.

In some possible embodiments, after generating a driving force by a quantitative fluid driving module to quantitatively transferring fluid from the fluid storage module into the flow channel, the method further includes: generate another driving force by the quantitative fluid driving module to quantitatively drive the fluid from the flow channel to the fluid storage module or to quantitatively transfer the fluid from the flow channel into a pipeline between the flow channel and the fluid storage module.

In some possible embodiments, the another driving force is a positive pressure driving force.

In some possible embodiments, after generating the driving force by a quantitative fluid driving module to quantitatively transfer fluid from the fluid storage module into the flow channel, the method further comprising, the method further includes: adjust a temperature in the flow channel through the fluid utilization module to cause a biochemical reaction of the fluid in the flow channel.

In some possible embodiments, the fluid transfer system further includes a waste liquid storage module, after adjusting the temperature in the flow channel through the fluid utilization module to cause the biochemical reaction of the fluid in the flow channel, the method further includes: generate another driving force by the quantitative fluid driving module to transfer the fluid from the flow channel to the waste liquid storage module.

In some possible embodiments, the waste liquid storage module is connected to the quantitative fluid driving module; wherein transfer the reacted fluid from the flow channel to the waste liquid storage module includes: generate the driving force as a negative pressure driving force by the quantitative fluid driving module to transfer the reacted fluid from the flow channel to the quantitative fluid driving module; and generate the driving force as a positive pressure driving force by the quantitative fluid driving module to transfer the reacted fluid from the quantitative fluid driving module to the waste liquid storage module.

In some possible embodiments, the fluid utilization module includes a plurality of flow channels, a size of each of the plurality of flow channels is same, such that a time for the fluid to flow from the fluid storage module into each of the plurality of flow channels is same.

In a third aspect, a biochemical substance analysis system is provided. The biochemical substance analysis system includes the fluid transfer system mentioned above.

The fluid transfer system provided by the present disclosure, through the cooperation of the pressure control module and the quantitative fluid driving module, constructs the positive pressure state at the inlet of the flow channel and applies the driving force for fluid movement at the outlet of the flow channel 211. Thus, the risk of gas bubble formation from the fluid can be reduced, which is beneficial for improving the quality of biochemical reactions in the flow channel and improving the accuracy of quantitative fluid transfer. The structural complexity of the fluid transfer system is simple and the cost is low. There is no need to add a degassing device, further simplifying the structural complexity of the fluid transfer system and reducing costs. By constructing a continuous positive pressure state at the inlet of the flow channel, the ultimate vacuum generated is not limited to one standard atmosphere, allowing more flexible adjustment of the fluid flow rate to adapt to different usage requirements. The fluid transfer process can effectively avoid cross-contamination between different types of fluids, reducing the need for isolation between different fluids, further simplifying the system structure and improving fluid transfer efficiency. The pressure source applied by the pressure control module is adjustable, allowing pressure adjustment based on the flow resistance in the flow channel, ensuring consistent fluid volume per pass through the flow channel. Thereby the consistency of each biochemical reaction time is improved. By connecting the waste liquid storage module with the quantitative fluid driving module, the waste liquid discharge problem can be effectively solved.

### BRIEF DESCRIPTION OF THE DRAWINGS

Implementations of the present technology will now be described, by way of embodiment, with reference to the attached figures. Obviously, the drawings are only some embodiments of the present disclosure. For those of ordinary skill in the art, other drawings can be obtained based on these drawings without creative work.

FIG. 1 is a block diagram of a fluid transfer system according to an embodiment of the present disclosure.

FIG. 2 is a diagrammatic view of a fluid transfer system according to an embodiment of the present disclosure.

FIG. 3 is a flowchart of a method for fluid transfer according to an embodiment of the present disclosure.

FIG. 4 is a diagrammatic view of a fluid transfer system according to another embodiment of the present disclosure.

FIG. 5 is a block diagram of a fluid transfer system according to another embodiment of the present disclosure.

FIG. 6 is a diagrammatic view of a fluid transfer system according to yet another embodiment of the present disclosure.

FIG. 7 is a block diagram of a biochemical substance analysis system according to an embodiment of the present disclosure.

### SYMBOL DESCRIPTION OF MAIN COMPONENTS

fluid transfer system 100, 200, 300; positive pressure source 311, 311a
fluid storage module 10, 10a; negative pressure source 312, 312a
fluid storage device 101, 101a; gas reservoir 302
container 111, 111a; positive pressure gas reservoir 321
control valves 112; negative pressure gas reservoir 322
fluid temporary storage device 102; second pressure monitoring device 303
temporary storage pool 121; switching valve 304
liquid level monitoring device 122; quantitative fluid driving module 40
first pressure monitoring device 123; fluid driving device 401
fluid utilization module 20; passage 411
fluid utilization device 201; fluid monitoring device 402
flow channel 211; flow direction switching device 403
inlet hole 212; waste liquid storage module 50
outlet hole 213; control module 60
inlet passage 214; biochemical substance analysis system 1000
outlet passage 215; imaging detection module 400
common portion 216; housing 500
temperature control device 202; inlet A
pressure control module 30, 30a; outlet B
pressure source 301, 301a.

### DETAILED DESCRIPTION

Implementations of the disclosure will now be described, by way of embodiments only, with reference to the drawings. The described embodiments are only portions of the embodiments of the present disclosure, rather than all the embodiments. The disclosure is illustrative only, and changes may be made in the detail within the principles of the present disclosure. It will, therefore, be appreciated that the embodiments may be modified within the scope of the claims.

It should be noted that when a component is referred to as being “fixed to” or “mounted on” another component, the component can be directly in contact with another component or a middle component may exist therebetween. When a component is considered to be “arranged on” another component, the component can be directly on another component or a middle component may exist therebetween. The term “and/or” as used herein means any combinations of one or more related listed items.

### Embodiment 1

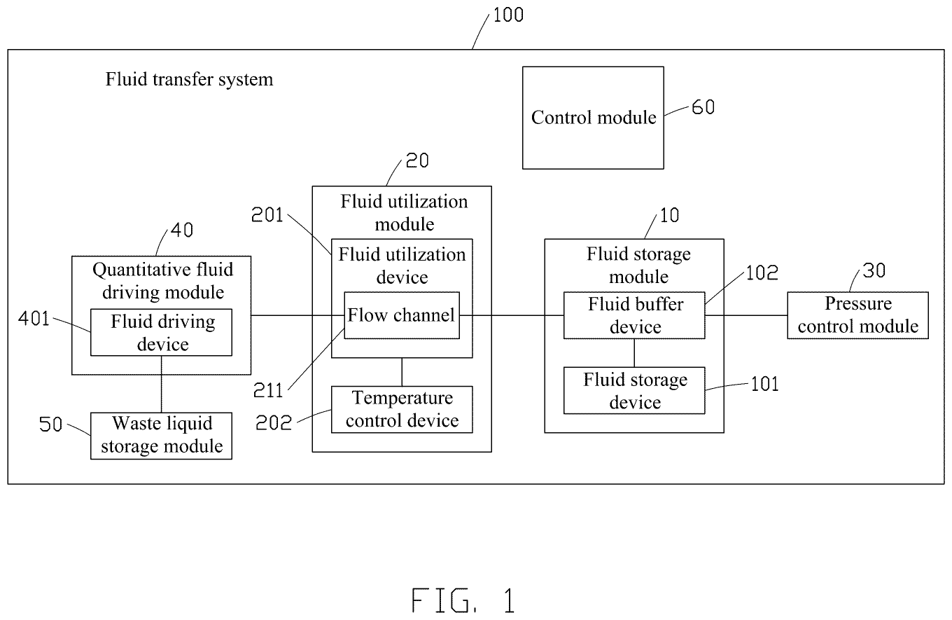

Referring to FIG. 1, a fluid transfer system 100 is provided according to an embodiment of the present disclosure. The fluid transfer system 100 can be used for quantitative transfer of fluids. The fluid can be in form of liquid or gas. In some embodiments, the fluid transfer system 100 can be used for quantitative transfer of fluids in biochemical substance analysis processes, for example, for quantitative transfer of biological samples and various reagents during gene sequencing analysis. The biological samples can be human blood samples, tissue samples, and saliva samples. The fluid transfer system 100 can also be used for fluid transfer in other experimental processes in biochemical laboratories.

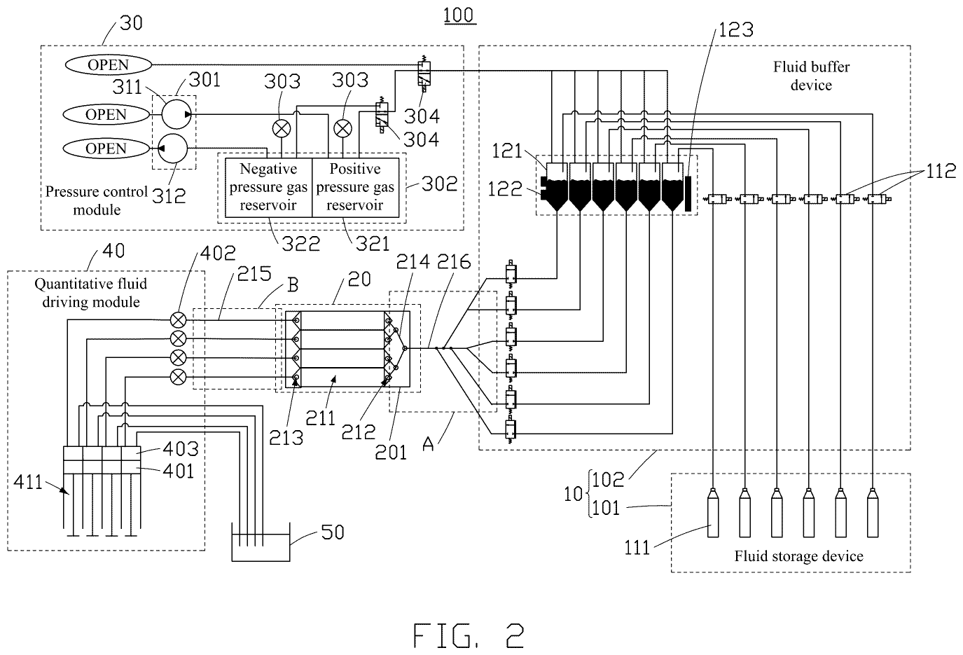

Referring to FIGS. 1 and 2, the fluid transfer system 100 includes a fluid storage module 10, a fluid utilization module 20, a pressure control module 30, and a quantitative fluid driving module 40. The fluid storage module 10 is used for storing fluids. In some embodiments, the fluid may include biological samples and various reagents required for biochemical reactions. The fluid utilization module 20 includes a flow channel 211. The flow channel 211 includes an inlet A and an outlet B. The inlet A is connected to the fluid storage module 10. The outlet B is connected to the quantitative fluid driving module 40. The fluid storage module 10 is also connected to the pressure control module 30. The pressure control module 30 is used to apply pressure to the fluid storage module 10 to maintain a positive pressure state at the inlet A of the flow channel 211. The quantitative fluid driving module 40 is used to generate a driving force to quantitatively transfer the fluids located in the fluid storage module 10 into the flow channel 211. That is, by establishing a positive pressure state at the inlet A of the flow channel 211 through the pressure control module 30 and applying the driving force at the outlet B of the flow channel 211 through the quantitative fluid driving module 40, quantitative fluid may be added into the flow channel 211, thereby allowing the flow channel 211 to maintain a positive or slightly negative pressure while fluid is flowing within the flow channel 211.

Referring to FIGS. 1 and 2, the fluid storage module 10 includes a fluid storage device 101 and a fluid temporary storage device 102 connected to the fluid storage device. And each of the pressure control module 30 and the flow channel 211 is connected to the fluid temporary storage device 102. The pressure control module 30 is used to apply pressure to the fluid temporary storage device 102. The pressure may be negative pressure and positive pressure. In some embodiments, the pressure control module 30 can generate negative pressure to transfer the fluid from the fluid storage device 101 to the fluid temporary storage device 102. After the fluid is transferred into the fluid temporary storage device 102, the pressure control module 30 can also generate positive pressure to apply a positive pressure to the fluid temporary storage device 102, thereby maintaining a positive pressure state at the inlet A of the flow channel 211.

In some embodiments, the fluid storage device 101 includes a plurality of containers 111, which can be, but not limited to, reagent bottles or independent cavities within a reagent cassette. The fluids may include biological samples, reagents, cleaning solutions, or pure water. Each type of fluid can be stored in a separate one of the containers 111. The fluid storage device 101 also includes a plurality of control valves 112. Each container 111 is connected to the fluid temporary storage device 102 through a fluid pipeline. A control valve 112 is disposed on the fluid pipeline between each container 111 and the fluid temporary storage device 102, and used to control the connection and disconnection between the container 111 and the fluid temporary storage device 102. In some embodiments, the control valve 112 can be a solenoid valve. Depending on functional requirements, the fluid storage device 101 may further include a liquid level detection sensor, and an air purification device.

In some embodiments, the fluid temporary storage device 102 includes a plurality of temporary storage pools 121. Each temporary storage pool 121 has multiple ports. The multiple ports can be connected to the flow channel 211, the pressure control module 30, and the fluid storage device 101 through some fluid pipelines. The fluid temporary storage device 102 further includes a liquid level monitoring device 122 and a first pressure monitoring device 123, which are used to monitor the state of the fluid within the temporary storage pool 121. Furthermore, multiple valves or pumps can be disposed on each temporary storage pool 121, such that the temporary storage pool 121 can be selectively connected to the flow channel 211 or the fluid storage device 101.

Referring to FIGS. 1 and 2, the fluid utilization module 20 includes a fluid utilization device 201. The fluid utilization device 201 includes one or more flow channels 211. Each flow channel 211 has an inlet hole 212 and an outlet hole 213. The inlet hole 212 is connected to the fluid temporary storage device 102 of the fluid storage module 10 through an inlet passage 214. The outlet hole 213 is connected to the quantitative fluid driving module 40 through an outlet passage 215. In some embodiments, the fluid utilization device 201 can be a sequencing chip used for gene sequencing.

In some embodiments, the fluid utilization device 201 includes a plurality of flow channels 211. Each flow channel 211 includes one inlet passage 214 and one outlet passage 215. The multiple inlet passages 214 converge to form a common portion 216. The common portion 216 is connected to the fluid temporary storage device 102. Each outlet passage 215 is connected to the quantitative fluid driving module 40 through a fluid pipeline. In some embodiments, the inlet passage 214 of the flow channel 211, the common portion 216, and a fluid pipeline connecting the common portion 216 to the fluid temporary storage device 102 constitute the inlet A of the flow channel 211. The outlet passage 215 of the flow channel 211 constitutes the outlet B of the flow channel 211. The multiple outlet passages 215 may not converge together, allowing the quantitative fluid driving module 40 to independently drive the fluid in each flow channel 211. When there are multiple outlet passages 215, the outlet passages 215 can also converge into a single common end, thereby allowing the quantitative fluid driving module 40 to simultaneously drive the fluid in the multiple flow channels 211.

In some embodiments, when a size of each of the plurality of flow channels 211 is same, such that a time for the fluid to flow from the fluid storage module 10 into each of the plurality of flow channels 211 is same. That is, the time required for the fluid to enter into each flow channel 211 from the fluid temporary storage device 102 through the common portion 216 and the inlet passage 214 is same, ensuring consistency in fluid processing time within each flow channel 211.

In other embodiments, the sizes of the multiple flow channels 211 can also be different from each other. In this case, by adjusting the length or cross-sectional area of the inlet passage 214, or adjusting the driving speed or driving time of the quantitative fluid driving module 40, consistency in liquid entry time for the multiple flow channels 211 can be ensured.

In some embodiments, the fluid utilization module 20 further includes a temperature control device 202. The temperature control device 202 can adjust a temperature within the flow channel 211 of the fluid utilization device 201, thereby enabling biochemical reactions of the fluids within the flow channel 211.

Referring to FIGS. 1 and 2, the pressure control module 30 is used to apply a certain pressure to the fluid storage module 10. The pressure can be positive pressure, negative pressure, or atmospheric pressure. In an embodiment, the pressure control module 30 is used to apply negative pressure and positive pressure to the fluid temporary storage device 102. After the pressure control module 30 generates negative pressure, a negative pressure is formed within the temporary storage pool 121 of the fluid temporary storage device 102. At this time, the temporary storage pool 121 is connected to the container 111 of the fluid storage device 101, and the fluid from the container 111 enters into the temporary storage pool 121. Subsequently, the pressure control module 30 generates positive pressure, thereby causing a positive pressure to form within the temporary storage pool 121 of the fluid temporary storage device 102. Thus, the fluid temporary storage device 102 can temporary store the fluid while maintaining and stabilizing a certain positive pressure, which further maintains a certain positive pressure state at the inlet A of the flow channel 211.

In some embodiments, the pressure control module 30 includes at least one pressure source 301 and at least one gas reservoir 302. Each of the at least one pressure source 301 is connected to one gas reservoir 302, and the gas reservoir 302 is connected to the fluid temporary storage device 102. In some embodiments, the at least one pressure source 301 includes a positive pressure source 311 and a negative pressure source 312. The at least one gas reservoir 302 includes a positive pressure gas reservoir 321 connected to the positive pressure source 311 and a negative pressure gas reservoir 322 connected to the negative pressure source 312. In some embodiments, the positive pressure source 311 can be a positive pressure pump, and the negative pressure source 312 can be a vacuum pump. The pressure source 301, the gas reservoir 302, the temporary storage pool 121, and the container 111 correspond one-to-one. When there are multiple sets of pressure sources 301, gas reservoirs 302, temporary storage pools 121, and containers 111, the multiple sets can be controlled independently or simultaneously.

In some embodiments, the pressure control module 30 further includes a switching valve 304 connected between the gas reservoir 302 and the fluid temporary storage device 102. The switching valve 304 is used to selectively connect the positive pressure gas reservoir 321 or the negative pressure gas reservoir 322 to the temporary storage pool 121, to apply negative or positive pressure to the temporary storage pool 121.

In some embodiments, the pressure control module 30 further includes a second pressure monitoring device 303, which can monitor the air pressure within the gas reservoir 302. In some embodiments, each of the positive pressure gas reservoir 321 and the negative pressure gas reservoir 322 is applyd with a second pressure monitoring device 303. In some embodiments, the second pressure monitoring device 303 can be a pressure sensor.

An operational process of the pressure control module 30 includes the following steps.

During the process of adding liquid to the fluid temporary storage device 102, the negative pressure range in the negative pressure gas reservoir 322 is [P1, P2], where absolute pressure P1<P2. The negative pressure range can be set according to actual needs. The negative pressure source 312 is started to extract gas from the negative pressure gas reservoir 322. When the pressure sensor monitors that the pressure reaches the set lower limit P1, the negative pressure source 312 stops and maintains the pressure. After connecting the negative pressure gas reservoir 322 to the corresponding temporary storage pool 121, the gas from the negative pressure gas reservoir 322 flows into the corresponding temporary storage pool 121, creating a negative pressure within the temporary storage pool 121. The negative pressure draws the fluid from the corresponding container 111 into the temporary storage pool 121. During the fluid extraction process, the negative pressure in the negative pressure gas reservoir 322 is continuously consumed. The pressure sensor monitors the negative pressure in the negative pressure gas reservoir 322 in real time. When the negative pressure exceeds the upper limit P2, the negative pressure source 312 is started to further maintain the negative pressure in the negative pressure gas reservoir 322 within the range [P1, P2].

During the process of applying positive pressure to the fluid temporary storage device 102, the positive pressure range in the positive pressure gas reservoir 321 is [P3, P4], where absolute pressure P3<P4. The positive pressure range can be set according to actual needs. The positive pressure source 311 is started to apply a pressure to the positive pressure gas reservoir 321. When the pressure sensor monitors that the pressure reaches the set upper limit P4, the positive pressure source 311 stops and maintains the pressure. After connecting the positive pressure gas reservoir 321 to the corresponding temporary storage pool 121, the gas from the positive pressure gas reservoir 321 flows into the corresponding temporary storage pool 121, creating a positive pressure within the temporary storage pool 121. At this point, the quantitative fluid driving module 40 is started to quantitatively transfer the fluid from the temporary storage pool 121 to the flow channel 211. As the fluid continuously flows into the flow channel 211, the gas in the positive pressure gas reservoir 321 is continuously consumed. The pressure sensor monitors the positive pressure in the positive pressure gas reservoir 321 in real time. When the positive pressure falls below the lower limit P3, the positive pressure source 311 is started to further maintain the positive pressure in the positive pressure gas reservoir 321 within the range [P3, P4], thereby ensuring that the temporary storage pool 121 maintains a positive pressure within the range [P3, P4] during the liquid addition process to the flow channel 211, and further ensuring that the inlet A of the flow channel 211 always maintains a positive pressure state during the liquid addition process.

Referring to FIGS. 1 and 2, the quantitative fluid driving module 40 is used to generate the driving force to achieve fluid movement within the fluid transfer system 100 and to precisely quantify the fluid. One form of the driving force can be pressure, including negative pressure and positive pressure, depending on the direction of fluid transport. The quantitative fluid driving module 40 includes a fluid driving device 401. The fluid driving device 401 is connected to the flow channel 211 and can generate the driving force. Furthermore, the fluid driving device 401 can drive the fluid quantitatively. For example, a graduated syringe can be used as the fluid driving device 401 to transfer the fluid quantitatively. In some embodiments, the fluid driving device 401 can generate a negative pressure driving force to transfer the fluid quantitatively from the fluid storage module 10 to the flow channel 211. After the fluid has entered the fluid temporary storage device 102 and the fluid temporary storage device 102 maintains a positive pressure state, the fluid driving device 401 generates a negative pressure driving force to draw the fluid from the fluid temporary storage device 102 into the flow channel 211. Since the fluid temporary storage device 102 (or the inlet A of the flow channel 211) always maintains a positive pressure state during the liquid addition process to the flow channel 211, the negative pressure driving force generated by the fluid driving device 401 does not need to be very large to transfer the fluid from the fluid temporary storage device 102 to the flow channel 211. Compared to the traditional method of using negative pressure alone to draw liquid, the negative pressure driving force is smaller, which prevents gas from escaping from the fluid and helps improve the quality of biochemical reactions within the flow channel 211. Thus, an additional degassing device is not needed, which can simplify the structural complexity of the fluid transfer system 100 and reduce costs. Additionally, the ultimate vacuum generated by the traditional method is usually one standard atmosphere, which limits the fluid speed and increases fluid transfer time. In the fluid transfer system 100, during the liquid addition process to the flow channel 211, by constructing a continuous positive pressure state at the inlet “A” of the flow channel 211, the ultimate vacuum generated is not limited to one standard atmosphere, allowing more flexible adjustment of the fluid flow rate to adapt to different usage requirements.

In some embodiments, the fluid driving device 401 is further configured to generate a positive pressure driving force to quantitatively drive the fluid from the flow channel 211 back to the fluid storage module 10 or back into an intermediate pipeline. The intermediate pipeline is a pipeline between the flow channel 211 and the fluid storage module 10, such as the fluid pipeline or the common portion 216. That is, during the liquid addition process to the flow channel 211, excess fluid drawn into the flow channel 211 by the negative pressure driving force can be pushed back to the fluid temporary storage device 102 of the fluid storage module 10 or the intermediate pipeline, thereby improving the accuracy of quantitative liquid addition and allowing reagent recovery and reuse.

In some embodiments, the fluid driving device 401 is connected to the fluid utilization device 201, enabling precise quantification of the fluid and transporting the fluid to the flow channel 211 of the fluid utilization device 201. In some embodiments, the fluid driving device 401 has one passage 411. The fluid utilization module 20 includes one or more flow channels 211. Each flow channel 211 is connected to the passage 411. When there are multiple flow channels 211, the multiple flow channels 211 can be controlled simultaneously to transfer fluid. Alternatively, in other embodiments, the fluid driving device 401 includes multiple passages 411, and the fluid utilization module 20 includes multiple flow channels 211. The flow channels 211 and the passages 411 correspond one-to-one, meaning each flow channel 211 is connected to one passage 411. Each of the plurality of passages 411 can be controlled independently or controlled in a coordinated manner with other passages 411 of the plurality of passages 411 to drive the fluid in each flow channel 211.

In some embodiments, the fluid driving device 401 can be a hydraulic unit, such as a peristaltic pump, plunger pump, syringe pump, gear pump, or diaphragm pump. The fluid driving device 401 can also be a pressure power unit, such as a vacuum pump, pressure pipettor, diaphragm pump, or air compressor.

In some embodiments, the quantitative fluid driving module 40 further includes a fluid monitoring device 402. The fluid monitoring device 402 may include at least one of a pressure sensor, flow sensor, and bubble sensor.

Referring to FIGS. 1 and 2, the fluid transfer system 100 further includes a waste liquid storage module 50, used for storing or discharging all or part of the waste liquid from the fluid transfer system 100. The quantitative fluid driving module 40 is further configured to generate a driving force to transfer a reacted fluid from the flow channel 211 to the waste liquid storage module 50.

In some embodiments, the waste liquid storage module 50 is connected to the quantitative fluid driving module 40. The quantitative fluid driving module 40 is configured to generate a negative pressure driving force to transfer the reacted fluid from the flow channel 211 into the quantitative fluid driving module 40. Subsequently, the quantitative fluid driving module 40 is further configured to generate a positive pressure driving force to transfer the reacted fluid from the quantitative fluid driving module 40 to the waste liquid storage module 50, achieving the purpose of waste liquid discharge. In some embodiments, the passage 411 of the fluid driving device 401 is connected to the waste liquid storage module 50.

In some embodiments, the quantitative fluid driving module 40 further includes a flow direction switching device 403 connected to the fluid driving device 401. The flow direction switching device 403 is used to switch the direction of fluid movement, allowing fluid to be drawn into the passage 411 of the fluid driving device 401 and also allowing fluid to be discharged from the passage 411 into the waste liquid storage module 50. In some embodiments, the flow direction switching device 403 is a switching valve, which can be a solenoid valve, rotary valve, pressure directional valve, electrohydraulic directional valve, manual directional valve, piezoelectric valve, pinch valve, rotary valve, or rotary shear valve.

In some embodiments, the waste liquid storage module 50 may have one or more ports. The waste liquid storage module 50 can be disposed inside or outside a biochemical substance analysis system. Alternatively, the waste liquid storage module 50 may have a waste liquid port, which is used to discharge waste liquid into a laboratory waste liquid treatment system directly. The waste liquid storage module 50 may include one or more containers. Furthermore, depending on functional requirements, the waste liquid storage module 50 may include sensors for liquid monitoring, air purification devices, or secondary anti-overflow equipment.

Referring to FIG. 1, the fluid transfer system 100 further includes a control module 60. The control module 60 is used to control the coordinated operation of the fluid storage module 10, the fluid utilization module 20, the pressure control module 30, and the quantitative fluid driving module 40.

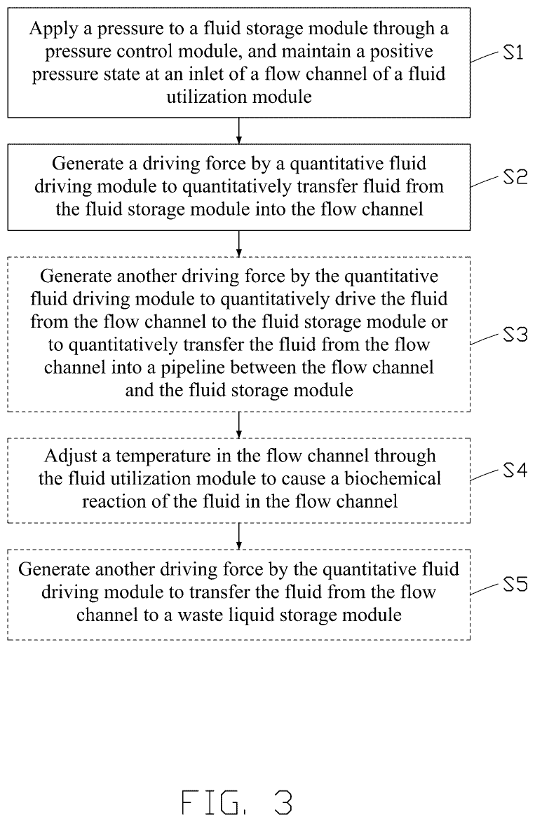

FIG. 3 illustrates a flowchart of a method for transferring fluid is provided according to the present disclosure. Referring to FIGS. 1 to 3, the method is provided by way of embodiment, as there are a variety of ways to carry out the method. The method can begin at block S1.

Block S1, a pressure to the fluid storage module 10 is applied by the pressure control module 30, and a positive pressure state at the inlet A of the flow channel 211 of the fluid utilization module 20 is maintained.

Block S1 includes the following steps:

Block S11, a negative pressure to the fluid temporary storage device 102 is applied by the pressure control module 30 to transfer a fluid from the fluid storage device 101 to the fluid temporary storage device 102.

Block S12, a pressure applied to the fluid temporary storage device 102 is positive pressure by the pressure control module 30 to maintain the positive pressure state at the inlet A of the flow channel 211.

Block S2, a driving force is generated by the quantitative fluid driving module 40 to quantitatively transfer the fluid located in the fluid storage module 10 into the flow channel 211.

A negative pressure driving force is generated by the quantitative fluid driving module 40 to quantitatively transfer the fluid from the fluid storage module 10 to the flow channel 211. In some embodiments, the negative pressure driving force is generated by the fluid driving device 401 to quantitatively transfer the fluid from the fluid temporary storage device 102 to the flow channel 211.

Block S3, another driving force is generated by the quantitative fluid driving module 40 to quantitatively drive the fluid from the flow channel 211 to the fluid storage module 10 or quantitatively drive the fluid from the flow channel 211 into the pipeline between the flow channel 211 and the fluid storage module 10.

A positive pressure driving force is generated by the fluid driving device 401 to quantitatively reflux the fluid from the flow channel 211 to the fluid temporary storage device 102 or quantitatively reflux the fluid from the flow channel 211 into the pipeline between the flow channel 211 and the fluid temporary storage device 102.

Block S4, a temperature in the flow channel 211 is adjusted by the fluid utilization module 20 to cause a biochemical reaction of the fluid within the flow channel 211.

The temperature in the flow channel 211 is adjusted by the temperature control device 202 to cause corresponding biochemical reactions of the fluid within the flow channel 211, such as sequencing reactions.

Block S5, a driving force is generated by the quantitative fluid driving module 40 to transfer the reacted fluid from the flow channel 211 to the waste liquid storage module 50.

Block S5 includes the following steps:

Block S51, a negative pressure driving force is generated by the quantitative fluid driving module 40 to transfer the reacted fluid from the flow channel 211 into the quantitative fluid driving module 40. That is, a negative pressure driving force is generated by the fluid driving device 401 to transfer the reacted fluid from the flow channel 211 into the passage 411 of the fluid driving device 401.

Block S52, a positive pressure driving force is generated by the quantitative fluid driving module 40 to transfer the reacted fluid from the quantitative fluid driving module 40 to the waste liquid storage module 50. That is, the flow direction switching device 403 is controlled to connect the passage 411 of the fluid driving device 401 to the waste liquid storage module 50, then the positive pressure driving force is generated by the fluid driving device 401 to discharge the reacted fluid from the passage 411 into the waste liquid storage module 50.

Referring to FIGS. 1 and 2, the method for fluid transfer is further illustrated below, mainly including a liquid addition process of the fluid temporary storage device 102 and a liquid addition process of the fluid utilization module 20.

The liquid addition process of the fluid temporary storage device 102 includes the following steps:

Step 1), the fluid temporary storage device 102 is connected to the fluid storage device 101.

Step 2), the pressure control module 30 is switched to the negative pressure source 312.

Step 3), the fluid temporary storage device 102 is connected to the negative pressure gas reservoir 322 of the pressure control module 30.

Step 4), the fluid is driven by the negative pressure to flow from the fluid storage device 101 to the fluid temporary storage device 102.

Step 5), the fluid temporary storage device 102 is disconnected from the fluid storage device 101 when the liquid level monitoring device 122 in the fluid temporary storage device 102 detects that the liquid reaches a specified position.

The liquid addition process of the fluid utilization module 20 includes the following steps:

Step 1), the pressure control module 30 is switched to the positive pressure source 311.

Step 2), the fluid temporary storage device 102 is connected to the positive pressure gas reservoir 321 of the pressure control module 30.

Step 3), the fluid temporary storage device 102 is connected to the flow channel 211 of the fluid utilization module 20.

Step 4), the quantitative fluid driving module 40 is connected to the flow channel 211. The negative pressure driving force is generated by the quantitative fluid driving module 40 to quantitatively draw fluid from the fluid temporary storage device 102 into the flow channel 211.

Step 5), the reacted fluid is drawn by the quantitative fluid driving module 40 from the flow channel 211 into the fluid driving device 401. After drawing the fluid, the fluid driving device 401 is switched to connect to the waste liquid storage module 50, and the reacted fluid is discharged into the waste liquid storage module 50.

In an embodiment, the method for fluid transfer is further illustrated as follows.

Step 1, a pressure range [P1, P2] is set for the negative pressure gas reservoir 322 (absolute pressure P1<P2).

Step 2, the negative pressure gas reservoir 322 is connected to the negative pressure source 312 (such as vacuum pump). Then the negative pressure source 312 is started to extract gas from the negative pressure gas reservoir 322. When the pressure detected by the second pressure monitoring device 303 reaches the set lower limit P1, the negative pressure source 312 is stopped and the pressure is maintained.

Step 3, the switching valve 304 (such as solenoid valve) is switched to the first position. The negative pressure gas reservoir 322 is connected to the corresponding temporary storage pool 121 via the fluid pipeline.

Step 4, the temporary storage pool 121 is connected to a corresponding container 111. At this time, the fluid in the container 111 will be drawn into the temporary storage pool 121 under negative pressure. The temporary storage pools 121 and the containers 111 correspond one-to-one. The solenoid valves between the temporary storage pools 121 and the corresponding containers 111 can be controlled independently or simultaneously.

Step 5, a liquid level in the temporary storage pool 121 is monitored through the liquid level monitoring device 122 in real time. When the liquid level reaches a set position, the solenoid valve between the temporary storage pool 121 and the corresponding container 111 is disconnected to stop liquid addition.

Step 6, during the liquid addition process, the negative pressure is continuously consumed. The pressure in the negative pressure gas reservoir 322 is monitored by the second pressure monitoring device 303 in real time. When the pressure exceeds the upper limit P2, the negative pressure source 312 is started to extract gas from the negative pressure gas reservoir 322. When the second pressure monitoring device 303 detects that the pressure reaches the set lower limit P1, the negative pressure source 312 is stopped and the pressure is maintained.

Step 7, a pressure range [P3, P4] is set for the positive pressure gas reservoir 321 (absolute PRESSURE P3<p4).

Step 8, the positive pressure gas reservoir 321 is connected to the positive pressure source 311 (such as positive pressure pump). The positive pressure source 311 is started to apply pressure to the positive pressure gas reservoir 321. When the second pressure monitoring device 303 detects that the pressure reaches the set upper limit P4, the positive pressure source 311 is stopped and the pressure is maintained.

Step 9, the switching valve 304 (such as solenoid valve) is switched to the second position. The positive pressure gas reservoir 321 is connected to the temporary storage pool 121 via the fluid pipeline.

Step 10, the positive pressure gas reservoir 321 is connected to the temporary storage pool 121, then the temporary storage pool 121 is connected to the flow channel 211.

Step 11, the flow direction switching device 403 (such as switching valve) of the fluid driving device 401 (such as syringe pump) is switched to connect to the flow channel 211. The fluid driving device 401 draws the fluid from the temporary storage pool 121 into the flow channel 211 according to the set flow rate. Simultaneously, the fluid driving device 401 can reversely push the drawn fluid quantitatively back to the temporary storage pool 121 or the intermediate pipeline. The temperature control device 202 is started to cause the corresponding biochemical reaction of the fluid within the flow channel 211.

Step 12, after the reaction is completed in the flow channel 211, the fluid driving device 401 draws the reacted waste liquid into the passage 411 of the fluid driving device 401. After completing the liquid drawing, the flow direction switching device 403 is switched to connect to the waste liquid storage module 50. The fluid driving device 401 discharges the drawn waste liquid into the waste liquid storage module 50. If necessary, the fluid driving device 401 can also reversely push the drawn fluid quantitatively back to the temporary storage pool 121 or the intermediate pipeline.

Step 13, during the fluid transport process, the gas in the positive pressure gas reservoir 321 is continuously consumed. The second pressure monitoring device 303 monitors the positive pressure in the positive pressure gas reservoir 321 in real time. When the positive pressure falls below the lower limit P3, the positive pressure source 311 is started to apply pressure to the positive pressure gas reservoir 321. When the second pressure monitoring device 303 detects that the pressure reaches the set upper limit P4, the positive pressure source 311 is stopped and the pressure is maintained.

The fluid transfer system 100 provided by the present disclosure, through the cooperation of the pressure control module 30 and the quantitative fluid driving module 40, constructs the positive pressure state at the inlet A of the flow channel 211 and applies the driving force for fluid movement at the outlet B of the flow channel 211. Thus, the risk of gas bubble formation from the fluid can be reduced, which is beneficial for improving the quality of biochemical reactions in the flow channel 211 and improving the accuracy of quantitative fluid transfer. The structural complexity of the fluid transfer system 100 is simple and the cost is low. There is no need to add a degassing device, further simplifying the structural complexity of the fluid transfer system 100 and reducing costs. By constructing a continuous positive pressure state at the inlet A of the flow channel 211, the ultimate vacuum generated is not limited to one standard atmosphere, allowing more flexible adjustment of the fluid flow rate to adapt to different usage requirements. The fluid transfer process can effectively avoid cross-contamination between different types of fluids, reducing the need for isolation between different fluids, further simplifying the system structure and improving fluid transfer efficiency. The pressure source applied by the pressure control module 30 is adjustable, allowing pressure adjustment based on the flow resistance in the flow channel 211, ensuring consistent fluid volume per pass through the flow channel 211. Thereby the consistency of each biochemical reaction time is improved. By connecting the waste liquid storage module 50 with the quantitative fluid driving module 40, the waste liquid discharge problem can be effectively solved.

### Embodiment 2

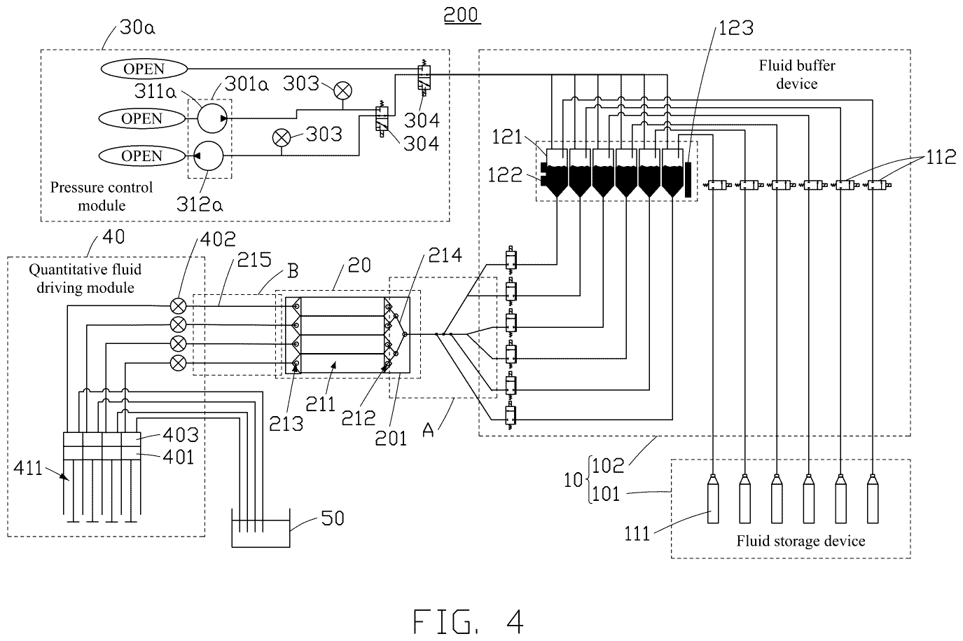

Referring to FIG. 4, a fluid transfer system 200 is further provided according to another embodiment of the present disclosure. A structure of the fluid transfer system 200 is substantially the same as that of the fluid transfer system 100 mentioned above. The main difference is that in the fluid transfer system 200, the pressure control module 30a omits the gas reservoir and includes a pressure source 301a. The pressure source 301a is directly connected to the fluid temporary storage device 102. A switching valve 304 is provided on a fluid pipeline between the pressure source 301a and the fluid temporary storage device 102.

In some embodiments, the pressure source 301a includes a positive pressure source 311a (such as positive pressure pump) and a negative pressure source 312a (such as vacuum pump). The fluid transfer system 200 directly applies air pressure to the temporary storage pool 121 of the fluid temporary storage device 102 through the positive pressure source 311a and the negative pressure source 312a.

Compared to the previous embodiment of the fluid transfer system 100, it can further simplify the structural complexity of the fluid transfer system 200 and reduce costs.

### Embodiment 3

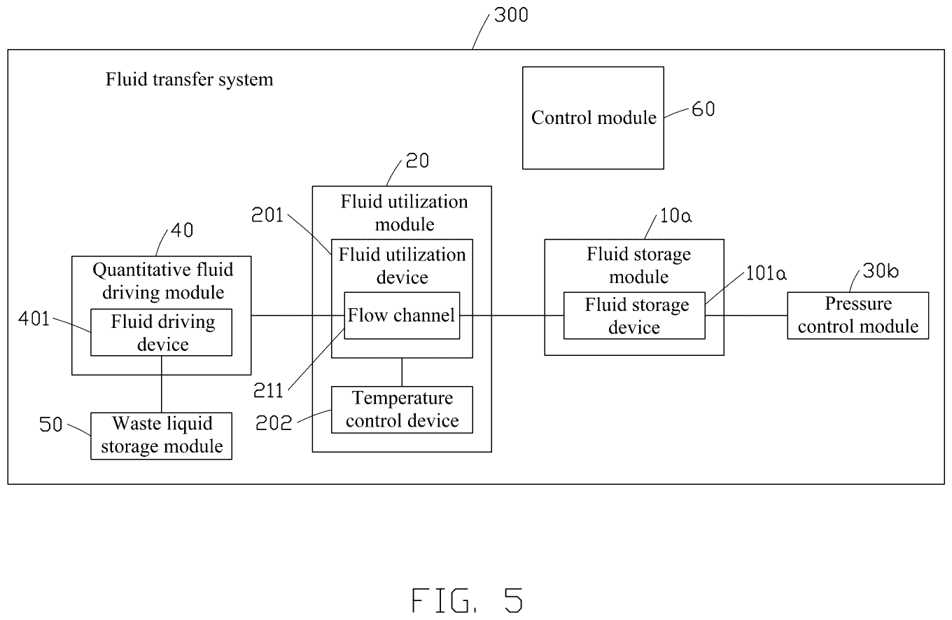

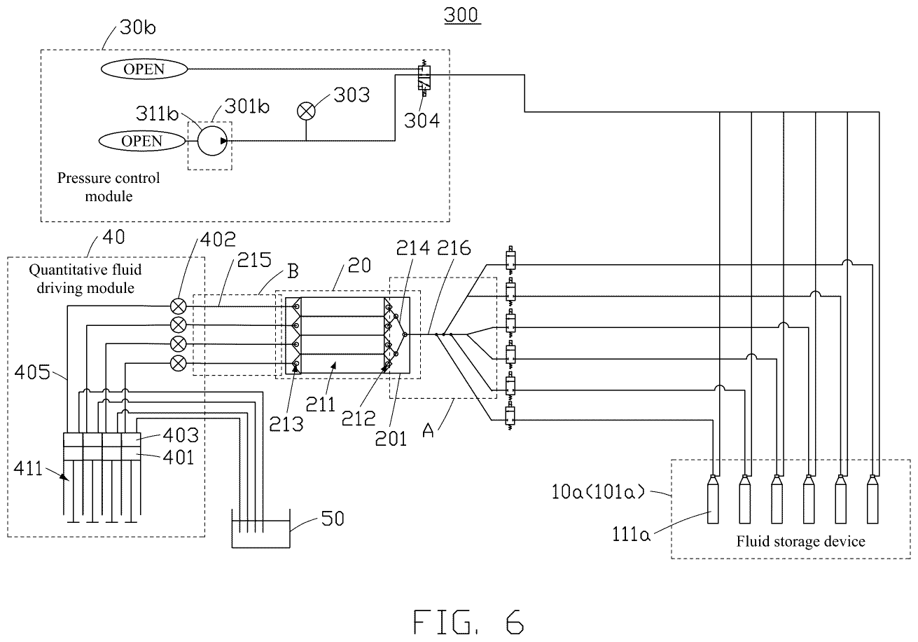

Referring to FIGS. 5 and 6, a fluid transfer system 300 is further provided according to yet another embodiment of the present disclosure. A structure of the fluid transfer system 300 is substantially the same as that of the fluid transfer system 100 mentioned above. The main difference is that in the fluid transfer system 300, the fluid storage module 10a includes a fluid storage device 101a. The pressure control module 30 is directly connected to the fluid storage device 101a of the fluid storage module 10a. The fluid storage device 101a is connected to the flow channel 211. The pressure control module 30b is used to apply pressure to the fluid storage device 101a. The pressure can be positive pressure, used to maintain a positive pressure state for the fluid storage device 101a, and further to maintain a positive pressure state at the inlet A of the flow channel 211.

The pressure control module 30b includes a pressure source 301b, a second pressure monitoring device 303, and a switching valve 304. The pressure source 301b includes a positive pressure source 311b. The positive pressure source 311b is connected to the fluid storage device 101a and is used to apply positive pressure to the fluid storage device 101a. In some embodiments, the fluid storage device 101a includes at least one container 111a for holding the fluid. The positive pressure source 311b is connected to the container 111a and is used to apply positive pressure inside the container 111a. In this case, the container 111a needs to be sealed to ensure the positive pressure does not leak.

Compared to fluid transfer using the fluid transfer system 100 mentioned above, when using the fluid transfer system 300 for fluid transfer, the process of adding liquid to the fluid temporary storage device is eliminated. Instead, the pressure control module 30 is used directly to apply positive pressure to the fluid storage device 101a, maintaining a positive pressure state for the fluid storage device 101a, thereby maintaining a positive pressure state at the inlet A of the flow channel 211. The subsequent steps are basically the same as the previous embodiment and will not be repeated here.

Compared to the previous embodiments, it can further simplify the structural complexity of the fluid transfer system 300, reduce costs, simplify the fluid transfer operation process, and improve fluid transfer efficiency.

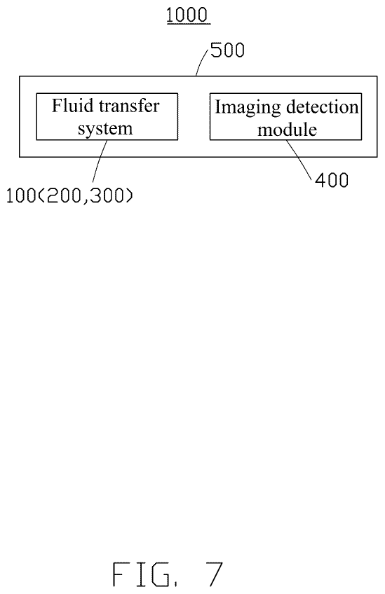

Referring to FIG. 7, a biochemical substance analysis system 1000 is further provided according to an embodiment of the present disclosure. The biochemical substance analysis system 1000 includes the fluid transfer system 100 (or 200, 300) as mentioned above.

In some embodiments, the biochemical substance analysis system 1000 can be a gene sequencer, further including an imaging detection module 400. The fluid utilization module 20 (such as sequencing chip) on which biochemical reactions are completed in the fluid transfer system 100 (or 200, 300) can be further transferred to the imaging detection module 400 for imaging detection analysis to complete the gene sequencing process. The transfer of the fluid utilization module 20 can be achieved through a robotic arm, moving track, or other transfer mechanism.

In some embodiments, the biochemical substance analysis system 1000 further includes a housing 500. The waste liquid storage module 50 of the fluid transfer system 100 (or 200, 300) can be located inside or outside the housing 500.

Even though information and advantages of the present embodiments have been set forth in the foregoing description, together with details of the structures and functions of the present embodiments, the disclosure is illustrative only. Changes may be made in detail, especially in matters of shape, size, and arrangement of parts within the principles of the present exemplary embodiments, to the full extent indicated by the plain meaning of the terms in which the appended claims are expressed.

1. A fluid transfer system, comprising:
a fluid storage module configured to store fluid;
a fluid utilization module defining a flow channel, wherein the flow channel comprises an inlet and an outlet, the inlet of the flow channel is connected to the fluid storage module;
a quantitative fluid driving module in connection with the outlet of the flow channel; and
a pressure control module in connection with the fluid storage module,
wherein the pressure control module is configured to apply a pressure to the fluid storage module and to maintain a positive pressure state at the inlet, and the quantitative fluid driving module is configured to generate a driving force to quantitatively transfer the fluid from the fluid storage module to the flow channel.

2. The fluid transfer system according to claim 1, wherein the fluid storage module comprises a fluid storage device and a fluid temporary storage device connected to the fluid storage device, and each of the pressure control module and the flow channel is connected to the fluid temporary storage device;
wherein the pressure control module is configured to apply the pressure to the fluid temporary storage device, the pressure comprises a negative pressure and a positive pressure, the negative pressure is configured to transfer the fluid from the fluid storage device to the fluid temporary storage device; and the positive pressure is applied to the fluid temporary storage device and configured to maintain the positive pressure state at the inlet of the flow channel.

3. The fluid transfer system according to claim 1, wherein the fluid storage module comprises a fluid storage device, the fluid storage device is connected to each of the pressure control module and the flow channel;
wherein the pressure control module is configured to apply the pressure to the fluid storage device, the pressure is a positive pressure, the positive pressure is configured to apply to the fluid storage device and maintain the positive pressure state at the inlet of the flow channel.

4. The fluid transfer system according to claim 2, wherein the pressure control module comprises:
at least one pressure source connected to the fluid storage device or to the fluid temporary storage device, wherein the at least one pressure source comprises a negative pressure source and a positive pressure source, the negative pressure source is configured to apply the negative pressure, and the positive pressure source is configured to apply the positive pressure;
at least one pressure monitoring device configured to monitor a pressure applied by the at least one pressure source; and
at least one switching valve disposed between the at least one pressure source and the fluid storage device or between the at least one pressure source and the fluid temporary storage device.

5. The fluid transfer system according to claim 4, wherein the pressure control module further comprises at least one gas reservoir disposed between the at least one pressure source and the at least one switching valve, the at least one gas reservoir is connected to the at least one pressure source and the fluid storage device or connected to the at least one pressure source and the fluid temporary storage device;
wherein the at least one gas reservoir comprises a negative pressure gas reservoir and a positive pressure gas reservoir, the positive pressure gas reservoir is connected to the positive pressure source, and the negative pressure gas reservoir is connected to the negative pressure source.

6. (canceled)

7. The fluid transfer system according to claim 1, wherein the quantitative fluid driving module comprises a fluid driving device in connection with the flow channel, wherein the fluid driving device is configured to generate the driving force as a negative pressure driving force to quantitatively transfer the fluid from the fluid storage module to the flow channel, and wherein the fluid driving device is further configured to generate the driving force as a positive pressure driving force to quantitatively transfer the fluid from the flow channel to the fluid storage module or to quantitatively transfer the fluid from the flow channel into a pipeline between the flow channel and the fluid storage module.

8-12. (canceled)

13. The fluid transfer system according to claim 1, wherein the quantitative fluid driving module comprises a plurality of passages, and the fluid utilization module comprises a plurality of flow channels, each of the plurality of flow channels is connected to a corresponding one of the plurality of passages, and each of the plurality of passages is configured to be controlled independently or controlled in a coordinated manner with other passages of the plurality of passages.

14. The fluid transfer system according to claim 13, wherein a size of each of the plurality of flow channels is same, such that a time for the fluid to flow from the fluid storage module into each of the plurality of flow channels is same.

15. The fluid transfer system according to claim 2, wherein the fluid temporary storage device comprises at least one temporary storage pool, and each of the pressure control module, the fluid storage device, and the flow channel is connected to the at least one temporary storage pool.

16. (canceled)

17. The fluid transfer system according to claim 1, wherein the fluid utilization module comprises a fluid utilization device defining the flow channel, and the fluid utilization device is a sequencing chip.

18. A method for fluid transfer, comprising:
applying a pressure to a fluid storage module through a pressure control module, and maintaining a positive pressure state at an inlet of a flow channel of a fluid utilization module; and
generating a driving force by a quantitative fluid driving module to quantitatively transfer fluid from the fluid storage module into the flow channel.

19. The method according to claim 18, wherein the fluid storage module comprises a fluid storage device and a fluid temporary storage device connected to the fluid storage device, and each of the pressure control module and the flow channel is connected to the fluid temporary storage device;
wherein applying the pressure to the fluid storage module comprises:
applying a negative pressure to the fluid temporary storage device through the pressure control module to transfer fluid from the fluid storage device to the fluid temporary storage device; and
applying a positive pressure to the fluid temporary storage device through the pressure control module and maintaining the positive pressure state at the inlet of the flow channel.

20. The method according to claim 18, wherein the fluid storage module comprises a fluid storage device connected to each of the pressure control module and the flow channel;
wherein applying the pressure to the fluid storage module comprises:
applying a positive pressure to the fluid storage device by the pressure control module, and maintaining a positive pressure state at the inlet of the flow channel.

21. The method according to claim 18, wherein the quantitative fluid driving module comprises a fluid driving device connected to the flow channel;
wherein generating the driving force comprises:
generating the driving force as a negative pressure driving force by the fluid driving device to quantitatively transfer the fluid from the fluid storage module to the flow channel.

22. The method according to claim 18, wherein after generating a driving force by a quantitative fluid driving module to quantitatively transferring fluid from the fluid storage module into the flow channel, the method further comprising:
generating another driving force by the quantitative fluid driving module to quantitatively drive the fluid from the flow channel to the fluid storage module or to quantitatively transfer the fluid from the flow channel into a pipeline between the flow channel and the fluid storage module.

23. (canceled)

24. The method according to claim 18, wherein after generating the driving force by a quantitative fluid driving module to quantitatively transfer fluid from the fluid storage module into the flow channel, the method further comprising:
adjusting a temperature in the flow channel through the fluid utilization module to cause a biochemical reaction of the fluid in the flow channel.

25. The method according to claim 24, wherein the fluid transfer system further comprises a waste liquid storage module, after adjusting the temperature in the flow channel through the fluid utilization module to cause the biochemical reaction of the fluid in the flow channel, the method further comprising:
generating another driving force by the quantitative fluid driving module to transfer the fluid from the flow channel to the waste liquid storage module.

26. (canceled)

27. The method according to claim 18, wherein the fluid utilization module comprises a plurality of flow channels, a size of each of the plurality of flow channels is same, such that a time for the fluid to flow from the fluid storage module into each of the plurality of flow channels is same.

28. (canceled)

29. The fluid transfer system according to claim 3, wherein the pressure control module comprises:
at least one pressure source connected to the fluid storage device, the at least one pressure source comprises a positive pressure source, the positive pressure source is configured to apply the positive pressure;
at least one pressure monitoring device configured to monitor a pressure applied by the at least one pressure source; and
at least one switching valve disposed between the at least one pressure source and the fluid storage device.

30. The fluid transfer system according to claim 29, wherein the pressure control module further comprises at least one gas reservoir disposed between the at least one pressure source and the at least one switching valve, wherein the at least one gas reservoir is connected to the at least one pressure source and the fluid storage device, and the at least one gas reservoir comprises a positive pressure gas reservoir connected to the positive pressure source.
